(19) United States
(12) Patent Application Publication
(10) Pub. No.: US 2005/0267372 A1
(43) Pub. Date: Dec. 1, 2005

(54) Catheter with improved illumination of the target region

(75) Inventor: Mathias Hörnig, Erlangen (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/142,757

(22) Filed: Jun. 1, 2005

(30) Foreign Application Priority Data
Jun. 1, 2004 (DE) 10 2004 026 619.0

(51) Int. Cl. A61B 8/14

(52) U.S. Cl. 600/462

(57) ABSTRACT

Catheter for examining and for performing interventions in cavities, in particular for medical applications in body cavities or vessels using a device for the improved illumination of the target region, wherein, besides at least one continuous central lumen (2), at least one lumen (4) is provided for receiving a glass fiber (9) in the catheter sheath (3).

### CROSS REFERENCE TO RELATED APPLICATIONS

This application claims priority to the German application No. 10 2004 026 619.0, filed Jun. 1, 2004 which is incorporated by reference herein in its entire ty.

### FIELD OF INVENTION

The invention relates to a catheter for examining and for performing interventions in cavities, in particular for medical applications in body cavities or vessels, using a device for the improved illumination of the target region.

### BACKGROUND OF INVENTION

Catheters are in the main hollow flexible instruments with which body cavities, e.g. vessels or cavities, can be penetrated, there being various forms and designs of catheter tube, ranging from the single lumen catheter to multilumen catheters and multilayer catheters. Thus, for example, catheters are already being developed for measuring pressure and for imaging the lumen of a vessel by means of an ultrasound head in the catheter tip. One problem in the use of new optical imagers is that of providing adequate flexible illumination of the cavities to be examined.

### SUMMARY OF INVENTION

The article “Imaging Catheter Gives Surgeons the Inside Picture” in S&TR, May 2000, refers to the possibility of arranging optical fibers in the walls of catheters, the optical fibers either being embedded in catheters obtainable on the market or being embedded in the walls of catheters during the process of extrusion of the catheter polymer. If the optical fibers are pointing forwards, i.e. oriented in an axial direction, then they will extend, as can be seen from the illustration on page 22 of the article, into the vascular space or generally into the surrounding space, without themselves being protected against external influences. Accordingly, the lumens accommodating the fibers will also be exposed to contamination and in general to contact with blood, etc.

In addition, where optical fibers are used to generate a radial light emission, as the illustration on page 22 of the article also shows, outlets are provided, as is clear from the image, so that the fibers and consequently also the lumens are not protected from the environment.

A cardiac catheter with an optimized probe is disclosed in U.S. 2003/0060867 A1, said cardiac catheter comprising a probe body in which optical fibers for irradiating pathological regions are provided in a mounting device. The guiding catheter 18 provided here has, as can be seen from FIG. 1, only a single lumen, a central arrangement of the optical fibers being achieved via a probe body 1 inside a plastic tube 17.

A multifunctional catheter system having multiple lumens is described in EP 1 145 731 A2. In it, as can be seen from FIG. 1, as well as a central lumen, a series of further axial lumens is also provided, the introduction of optical fibers or fiber bundles for illuminating a target tissue also being possible.

An object of the invention is therefore to design a catheter such that improved illumination of the intervention region in the area of the catheter tip is possible in a simple manner, without the aforementioned disadvantages.

This object is achieved by the claims.

In order to achieve this object, the invention provides that, as well as at least one continuous central lumen, at least one lumen for accommodating a glass fiber is provided in the catheter sheath, whereby in the preferred case of multiple glass fibers being used, these should usefully be spaced equidistantly around the periphery.

For the particularly preferred medical applications of catheters according to the invention, the diameters of the lumens equipped with glass fibers should preferably lie between 150 μm and 1 mm so as to ensure as small a catheter diameter as possible.

Light is fed, for example via a laser diode, into the glass fibers and into the cavity to be examined from a unit located at the catheter opening. The preferred axially symmetrical arrangement with multiple glass fibers integrated on the one hand enables particularly homogeneous illumination of the cavity to be examined and on the other hand keeps the central lumen clear for any imaging unit.

The catheter according to the invention may in addition have an optical imaging unit, for example a CCD or a CMOS, installed rigidly or adjustably in a further lumen, for control of which and/or for illumination of the cavity to be examined the glass fibers are used.

Finally, it also lies within the scope of the invention for the lumens for the glass fibers to be closed at the free insertion end of the catheter, said catheter consisting at least in this region of transparent material. With this type of integration of catheters into the glass-fiber sheath without an opening at the front end, particularly complex disinfection steps can be avoided where such catheters are used, since only the outside wall of the catheter has to be disinfected, but not the lumens for accommodating the glass fibers.

The advantage of a catheter equipped with glass fibers according to the invention lies not only in the fact that an integrated medium for illuminati ng the cavity to be examined is present, but also in the fact that, as a result of the isocentric arrangement of multiple glass fibers in the catheter, particularly homogeneous illumination can be achieved. In addition, where a catheter according to the invention is used, the introduction and removal of light sources for examinations can be dispensed with. The glass fibers can possess such great flexibility that they do not prevent nor even impede catheter movements, it being possible for the glass fibers to have such a small diameter that they can be integrated in the catheter without problems. The light intensity of the glass fibers can be appropriately adapted to requirements via a control unit, for example via various transmit diodes or potentiometer controls. Due to the insensitivity of glass fibers to external disturbances such as electromagnetism, the new types of catheter are particularly well suited for use in magnetic fields and also enable the problem-free transmission of signals, possibly at a high transmission rate, to and from a measuring instrument, imaging unit or such like located, for example, in the catheter tip.

### BRIEF DESCRIPTION OF THE DRAWINGS

Further advantages, features and details of the invention are set down in the description of some exemplary embodiments below and from the drawings, in which:

FIGS. 1 and 2 show cross-sections through catheters according to the invention comprising three or four glass fibers,

FIG. 3 shows a section through a further embodiment of a catheter according to the invention comprising separate glass fibers for control purposes,

FIG. 4 shows a cross-section through a further embodiment of a catheter according to the invention and

FIG. 5 shows a section through the catheter tip, along the line V-V in FIG. 4.

### DETAILED DESCRIPTION OF INVENTION

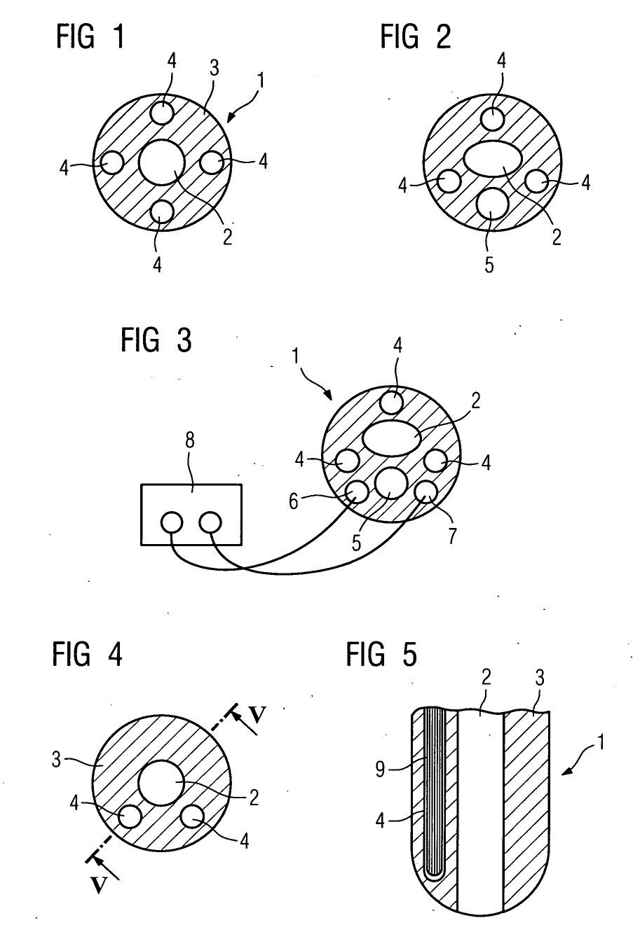

The catheter 1 in FIG. 1 contains, as well as the continuous central lumen 2, in the region of the catheter sheath 3 four lumens 4 for accommodating glass fibers, the size of these glass fibers essentially corresponding to the diameter of these lumens 4. As in this exemplary embodiment according to FIG. 1, in the exemplary embodiment according to FIG. 2, in which only three lumens 4 are provided for accommodating glass fibers and in which an additional lumen 5 is provided for accommodating an optical unit, the lumens for the glass fibers are also arranged essentially in an axially symmetrical manner.

FIG. 3 shows a section through a catheter 1, comprising a central lumen 2 and three axially symmetrically arranged lumens 4 for glass fibers, in which, besides the lumen 5 for the optical unit, for example a CCD camera or a CMOS, there are two further glass fibers 6 and 7 for the control (transmitter and receiver) of the optical unit with the aid of a control box 8.

FIGS. 4 and 5 show an embodiment of a catheter in which the lumens 4 for accommodating the glass fibers 9 are not continuous but are closed in the region of the catheter tip, while the central lumen 2 is of course open at the front. In conjunction with a design of the catheter from a material which is transparent at least in the region of the catheter tip, the required lighting effects can be achieved without difficulty using the glass fibers 9 in the lumens of the catheter sheath 3, while at the same time the disinfection of such catheters is considerably easier as a result of the absence of an opening of the lumens 4 at the catheter tip. The disinfection of very thin lumens with a diameter of from 150 μm to 1 mm is extremely complicated and in the case of this closed design as per FIGS. 4 and 5 can be fully dispensed with.

1-2. (canceled)

3. A catheter for examining and for performing interventions in a cavity, comprising:
a free catheter end for inserting the catheter into the cavity, the catheter made of a transparent material at least at the free catheter end;
an illumination device including at least a first and a second glass fiber for illuminating a target area included in the cavity, the second glass fiber having an inserting end arranged adjacent to the free catheter end;
a catheter sheath for accommodating the catheter;
a central continuous lumen for accommodating the first glass fiber, the central lumen arranged within the catheter sheath and passing through the catheter sheath; and
at least one further lumen for accommodating the second glass fiber, the at least one further lumen arranged within the catheter sheath, wherein the at least one further lumen:
passes through at least part of the catheter sheath, and
is closed at the inserting end.

4. The catheter according to claim 3, wherein the catheter is a medical catheter, and the cavity is a body cavity of a patient.

5. The catheter according to claim 3, wherein the catheter is a medical catheter, and the cavity includes a vessel of a patient.

6. The catheter according to claim 3, wherein the second glass fiber includes a plurality of glass fibers distributed about a cross-section of the catheter.

7. The catheter according to claim 6, wherein the plurality of glass fibers are equidistantly distributed.

8. The catheter according to claim 7, wherein the plurality of glass fibers are arranged within an outer ring section of the cross-section.
